(19) United States
(12) Patent Application Publication
(10) Pub. No.: US 2026/0001966 A1
(43) Pub. Date: Jan. 1, 2026

(54) Medical Use of Functionalized Chitosan

(71) Applicants: MEXBRAIN, Villeurbanne (FR); UNIVERSITE CLAUDE BERNARD LYON 1, Villeurbanne (FR); CENTRE NATIONAL DE LA RECHERCHE SCIENTIFIQUE, Paris (FR); UNIVERSITE JEAN MONNET SAINT-ETIENNE, Saint Etienne (FR); INSTITUT NATIONAL DES SCIENCES APPLIQUEES DE LYON, Villeurbanne (FR)

(72) Inventors: Coralie Grange, Villeurbanne (FR); Axel Aigle, Bron (FR); Thomas Brichart, Villeurbanne (FR); Olivier Tillement, Fontaines Saint-Martin (FR); François Lux, Villeurbanne (FR); Laurent David, Lyon (FR); Victor Ehrlich, Levallois Perret (FR); Thomas Grea, Lyon (FR); Juan Salazar Ariza, Villeurbanne (FR)

(73) Assignees: MEXBRAIN, Villeurbanne (FR); UNIVERSITE CLAUDE BERNARD LYON 1, Villeurbanne (FR); CENTRE NATIONAL DE LA RECHERCHE SCIENTIFIQUE, Paris (FR); UNIVERSITE JEAN MONNET SAINT-ETIENNE, Saint Etienne (FR); INSTITUT NATIONAL DES SCIENCES APPLIQUEES DE LYON, Villeurbanne (FR)

(21) Appl. No.: 18/880,666

(22) PCT Filed: Jul. 3, 2023
(86) PCT No.: PCT/EP2023/068258

§ 371 Date: Jan. 2, 2025

(30) Foreign Application Priority Data
Jul. 4, 2022 (EP) 22305996.5

(51) Int. Cl. C08B 37/08; A61K 9/00; A61K 31/722; A61P 29/00

(52) U.S. Cl. CPC C08B 37/003; A61K 9/0019; A61K 31/722; A61P 29/00

(57) ABSTRACT

The present disclosure relates to a functionalized statistic chitosan of weight average molecular mass between 100 kDa and 1000 kDa and of formula (I):

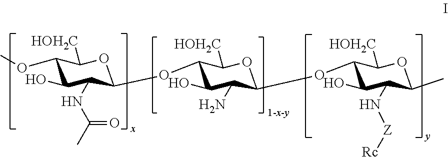

wherein
each Rc is independently a moiety comprising a chelating agent,
each Z is independently a linker which is a single bond or a hydrocarbon chain containing between 1 and 12 carbon atoms, said hydrocarbon chain is linear or branched and optionally contains one or more unsaturations and one or more heteroatoms,
x is between 0.005 and 0.7, preferably between 0.05 and 0.7,
y is between 0.01 and 0.7, preferably between 0.05 and 0.2,
the ratio y/x being greater than or equal to 0.05, preferably greater than or equal to 0.15, and
the sum x+y being greater than or equal to 0.15, for use in the treatment of diseases characterized by local inflammation induced by and/or inducing a deregulation of metal homeostasis and increased oxidative stress, in a subject in need thereof.

### FIELD OF THE INVENTION

The present invention relates to medical use of functionalized chitosan. In particular, the invention relates to a functionalized chitosan with statistic macromolecular structure for use in the treatment of diseases characterized by local inflammation induced by and/or inducing a dysregulation of metal homeostasis, and increased oxidative stress, in a subject in need thereof.

### BACKGROUND

Metal accumulation in the body is a clinical issue involved in several pathologies: metal poisoning alone causes each year over 1 million deaths and costs over 1.4% of the global gross domestic product (Mohamed I. Elsaid et al. Health care utilization and economic burdens of hemochromatosis in the united states: a population-based claims study. Eng. Journal of Managed Care & Specialty Pharmacy 25:12 (2019); Philippe Grandjean and Martine Bellanger. Calculation of the disease burden associated with environmental chemical exposures: application of toxicological information in health economic estimation. Environmental Health 16:1 (2017)). Moreover, there are pathologies related to the dysregulation of essential elements: the best known are genetic diseases like Wilson disease for copper or hemochromatosis for iron.

During the last few years, the clinical research has focused its attention on the link between the dysregulation of essential metals homeostasis and the onset of neurodegenerative proteinopathies such as Parkinson's disease and Alzheimer's disease. Indeed, an accumulation of essential metals is found in correspondence of the neurotoxic protein deposits typical of these pathologies: metal cations are probably involved in the modification of the morphology and the solubility properties of those proteins. Furthermore, the presence of these cations can induce the generation of reactive oxygen species (ROS), for example by Fenton reaction, inducing inflammation and accelerating the degeneration of these conditions. Several clinical trials, aimed to treat neurodegenerative diseases through chelation therapy to target iron accumulation, further support these hypotheses and already showed promising results (A Pilot Clinical Trial With the Iron Chelator Deferiprone in Parkinson's Disease—Full Text View—ClinicalTrials.gov.; Efficacy and Safety of the Iron Chelator Deferiprone in Parkinson's Diseasez—Full Text view—ClinicalTrials.gov.; Lille University Hospital, European Commission, and ApoPharma. Conservative Iron Chelation as a Disease-modifying Strategy in Parkinson's Disease. European Multicenter, parallel-group, Placebo-controlled, Randomized Clinical Trial of Deferiprone”; Neuroscience Trials Australia. Deferiprone to Delay Dementia (The 3D Study): a Clinical proof of Concept Study).

Metals have also an important impact in a lot of local inflammations.

A large body of evidence indicates that dysregulation of brain iron metabolism and transport plays a role in mediating neuronal damage after ischemic stroke. After intracerebral hemorrhage, hemoglobin breakdowns releasing iron, this iron overload in the brain causes brain injury that can be aggravated by the inflammatory response (Xi G, Keep R F, Hoff J T. Mechanisms of brain injury after intracerebral hemorrhage (Lancet Neurol. 2006 January; 5 (1): 53-63). Iron-mediated toxicity continues to damage the neurons for several days to weeks after stroke onset, and drugs that modify iron metabolism can reduce stroke lesion volume, neuronal necrosis, brain injury, and cerebral edema in animal stroke models (Magdy H. Selim, Rajiv R. Ratan, Ageing Research Reviews 3 (2004) 345-353).

Iron storage is also statistically significantly increased in peritoneal macrophages of patients with endometrioses and correlates with iron overload in peritoneal fluids. Indeed, iron accumulation in macrophages leads to excessive production of ROS and enhanced activation of NF-kB. This transcriptional factor has been implicated in endometriosis and shown to induce expression of multiple genes encoding proinflammatory cytokines, growth and angiogenic factors, adhesion molecules and inducible enzymes which are well expressed by activated peritoneal macrophages (Lousse et al. Iron and macrophages in endometriosis, Fertil Steril Vol. 91, No. 5, May 2009).

Another area of clinical interest relates to interstitial cystitis which is a chronic inflammatory condition of the bladder characterized by the absence of signs of infection but often a lot of micro-hemorrhage. Ener at al showed that the dysregulation of ROS production plays an important role in interstitial cystitis. Decrease of oxidative stress by antioxydants significantly reduced inflammation in a rat model of IC. (Georg Hagn, et al. Iron chelation as novel treatment for interstitial cystitis, Pharmacology 2019; 103:159-162)

In addition, iron storage throughout the body increases with age, associated with raising macular iron levels. Iron can then cause oxidative damage and contribute to retinal degeneration associated with several ocular diseases such as glaucoma or age-related macular degeneration (AMD) (He X, et al. Iron homeostasis and toxicity in retinal degeneration. Prog Retin Eye Res. 2007 November; 26 (6): 649-73). Therefore, finding a safe and efficient treatment to restore metal homeostasis is a current challenge in many diseases characterized by local inflammatory, especially inflammation in a cavity. Metals must be rapidly chelated in a safe way and without removal of other essential elements. The standard treatment nowadays for heavy metallic deregulation is the chelation therapy administered systemically or the phlebotomy. Chelation therapy has proven its efficacy in some specific cases but remains limited to specific pathologies. In the framework of Wilson's disease where it is traditionally used, for example, chelatant use is characterized by a relatively short lifespan in the body, a high-dose cytotoxicity and quite severe side effects, such as liver or kidney damage and irreversible neurological damage. With the current treatments, between 30% and 40% of the patients experience adverse effects, which makes 50% of them non-eligible for this kind of treatment, including pregnant women, elderly, children and chronic kidney disease (CKD) patients. Moreover, 56% of the patients in treatment are non-compliant because of the elevated number of pills they have to take during the day, making the treatment less effective. Furthermore, between 20% and 30% of the patients worsen their condition anyway over time (M. J. Kosnett. Chelation for heavy metals (arsenic, lead, and mercury): protective or perilous? Clinical Pharmacology & Therapeutics 88:3 (2010)). For patients presenting local inflammation due to metallic ions, systemic chelation therapy cannot be used due to insufficient benefice to risk ratio and a more localized treatment is critically needed.

WO2019/122790 discloses a medical device introducible into the body for the maintenance of metal homeostasis for therapeutic purposes comprising a chelating agent for extracting metals.

WO2022/023677 describes a statistical polysaccharide with a weight-average molecular weight of between 100 kDa and 1000 kDa and its use in a dialysis process in order to capture at least one metal, in an MRI imaging process, in a brachytherapy process or in a process for marking foodstuffs to prevent forgeries.

It remains therefore a challenge to design a safe and efficient local treatment which meets this clinical need of restoring metal homeostasis in diseases characterized by local inflammation.

### SUMMARY OF THE INVENTION

A first aspect of the disclosure relates to a functionalized statistic chitosan of weight average molecular mass between 100 kDa and 1000 kDa and of formula (I):

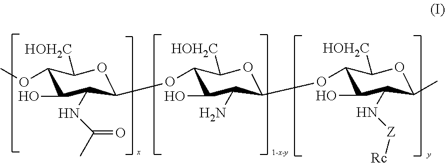

wherein
each Rc is independently a moiety comprising a chelating agent,
each Z is independently a linker which is a single bond or a hydrocarbon chain containing between 1 and 12 carbon atoms, said hydrocarbon chain is linear or branched and optionally contains one or more unsaturations and one or more heteroatoms, preferably chosen from nitrogen, oxygen, sulfur and halogens,
x is between 0.005 and 0.7, preferably between 0.05 and 0.7,
y is between 0.01 and 0.7, preferably between 0.05 and 0.2,
the ratio y/x being greater than or equal to 0.05, preferably greater than or equal to 0.15, and
the sum x+y being greater than or equal to 0.15, preferably greater than or equal to 0.30, more preferably greater than or equal to 0.35,
for use in the treatment of diseases characterized by local inflammation induced by and/or inducing a deregulation of metal homeostasis and increased oxidative stress, in a subject in need thereof.

A second aspect of the disclosure relates to an injectable solution comprising a functionalized statistic chitosan of weight average molecular mass between 100 kDa and 1000 kDa and of formula (I):

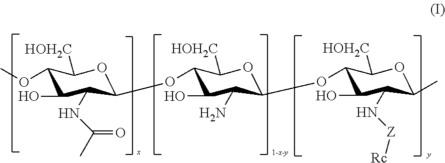

wherein
each Rc is independently a moiety comprising a chelating agent,
each Z is independently a linker which is a single bond or a hydrocarbon chain containing between 1 and 12 carbon atoms, said hydrocarbon chain is linear or branched and optionally contains one or more unsaturations and one or more heteroatoms, preferably chosen from nitrogen, oxygen, sulfur and halogens,
x is between 0.005 and 0.7, preferably between 0.05 and 0.7, and more preferably between 0.2 and 0.6,
y is between 0.01 and 0.7, preferably between 0.05 and 0.2,
the ratio y/x being greater than or equal to 0.05, preferably greater than or equal to 0.15, and
the sum x+y being greater than or equal to 0.15, preferably greater than or equal 0.30, more preferably greater than or equal to 0.35,
and one or more pharmaceutically acceptable excipients,
wherein the concentration of said functionalized chitosan is of between 10 g/L and 50 g/L, preferably between 15 g/L and 30 g/L, more preferably between 18 g/L and 25 g/L, even more preferably around 20 g/L.

### DETAILED DESCRIPTION OF THE INVENTION

### Definitions

In the following, terms as used herein are defined in their meaning.

The term “about” or “ca.” has herein the meaning that the following value may vary for ±20%, preferably ±10%, more preferably ±5%, even more preferably ±2%, even more preferably ±1%.

Unless otherwise defined, “%” has herein the meaning of weight percent (wt %), also referred to as weight by weight percent (w/w %).

The chelating agent in the context of the present disclosure may be
DOTA: 1,4,7,10-tetraazacyclododecane-N,N′,N″,N′″-teracetic acid;
NOTA: 1,4,7-triazacyclononane-1,4,7-triacetic acid;
NODAGA: 1,4,7-triazacyclononane-1-glutaric-4,7-diacetic acid;
DOTAGA: 2-(4,7,10-tris(carboxymethyl)-1,4,7,10-tetraazacyclododecan-1-yl)pentanedioicacid;
DOTAM: 1,4,7,10-tetrakis(carbamoylmethyl)-1,4,7,10-tetraazacyclodecane;
NOTAM: 1,4,7-tetrakis(carbamoylmethyl)-1,4,7-triazacyclononane;
DOTP: 1,4,7,10-tetraazacyclododecane 1,4,7,10-tetrakis(methylene phosphonate;
NOTP: 1,4,7-tetrakis(methylene phosphonate)-1,4,7-triazacyclononane;
TETA: 1,4,8,11-tetraazacyclotetradecane-N,N′,N″,N′-teracetic acid;
TETAM: 1,4,8,11-tetraazacyclotetradecane-N,N′,N″,N″-tetrakis(carbamoyl methyl);
DTPA: diethylene triaminopentaacetic acid;
Bz-DFO: benzyl deferoxamine;
DFO: deferoxamine.

As used herein, the terms “effective amount” or “therapeutically efficient amount” of a compound refer to an amount of the compound that will induce the biological or medical response of a subject, for example, ameliorate the symptoms, alleviate conditions, slow or delay disease progression, or prevent a disease.

As used herein, the term “independently” for the Rc moiety i.e. the chelating agent, means that each Rc of the functionalized statistic chitosan of formula (I) is a chelating agent that may be different from one to another, or may be identical. For example, each Rc is identical i.e., there is one type of Rc throughout the functionalized statistic chitosan, or there may be more than one type of Rc throughout the functionalized statistic chitosan i.e. two, three, four, five or even n different Rc, n being an integer.

As used herein “combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration, the terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

### The Functionalized Statistic Chitosan

The functionalized chitosan has a statistic macromolecular structure.

In a first aspect of the disclosure relates to a functionalized statistic chitosan of weight average molecular mass between 100 kDa and 1000 kDa and of formula (I):

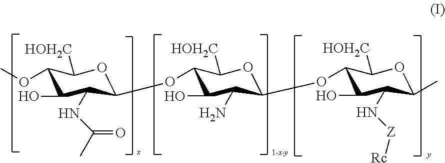

wherein
each Rc is independently a moiety comprising a chelating agent,
each Z is independently a linker which is a single bond or a hydrocarbon chain containing between 1 and 12 carbon atoms, said hydrocarbon chain is linear or branched and optionally contains one or more unsaturations and being able to contain one or more heteroatoms, preferably chosen from nitrogen, oxygen, sulfur and halogens,
x is between 0.005 and 0.7, preferably between 0.05 and 0.7,
y is between 0.01 and 0.7, preferably between 0.05 and 0.2,
the ratio y/x being greater than or equal to 0.05, preferably greater than or equal to 0.15, and
the sum x+y being greater than or equal to 0.15, preferably greater than or equal to 0.30, more preferably greater than or equal to 0.35,
for use in the treatment of diseases characterized by local inflammation induced by and/or inducing a deregulation of metal homeostasis and increased oxidative stress, in a subject in need thereof.

It is understood that, in the above formula I, more than one Rc group may be present in the functionalized statistic chitosan. These Rc groups may be the same or different from each other. They are all independently selected from the groups carrying a chelating agent. The same applies to the Z-linkers, several Z-linkers may be present, and they may be identical or different from each other.

In an embodiment, in formula I, x is between 0.005 and 0.6; y is between 0.1 and 0.9; the ratio y/x being greater than 0.16; and the sum x+y being greater than 0.30.

In a specific embodiment, the functionalized statistic chitosan of the present disclosure has a complexation constant of at least 1015 for a d or f transition element.

In an embodiment, the functionalized statistic chitosan of formula I is a functionalized statistic chitosan of formula (II):

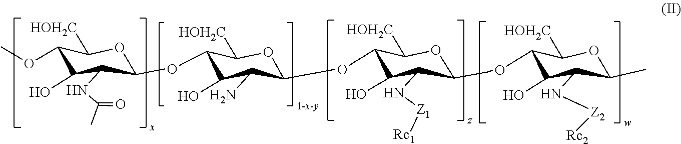

wherein
Rc1 and Rc2 are different, and are moiety comprising a chelating agent,
Z1 and Z2, identical or different, are linkers which are a single bond or a hydrocarbon chain containing between 1 and 12 carbon atoms, said hydrocarbon chain is linear or branched and optionally contains one or more unsaturations and one or more heteroatoms, preferably chosen from nitrogen, oxygen, sulfur and halogens,
x is between 0.005 and 0.7, preferably between 0.05 and 0.7, and more preferably between 0.2 and 0.6,
y=z+w is between 0.01 and 0.7, preferably between 0.05 and 0.2,
the ratio y/x being greater than or equal to 0.05, preferably greater than or equal to 0.15,
the sum x+y being greater than or equal to 0.15, preferably greater than or equal 0.30, more preferably greater than or equal to 0.35, and
z/y is between 0.5 and 1

In this embodiment, the functionalized statistic chitosan of formula (II) may comprise either a single type of moiety comprising a chelating agent, Rc1, when z is equal to 1, or 2 types of moieties comprising a chelating agent, Rc1 and Rc2, when 0.5≤z<1.

In an embodiment, z/y is between 0.8 and 0.99, Rc1 moiety is thus in the majority.

In another embodiment, in formula II, x is between 0.005 and 0.6; y is between 0.1 and 0.9; the ratio y/x being greater than 0.3; and z is between 0.5 and 1.

### Rc Moiety (Rc, Rc1 and Rc2)

As used herein, the term “Rc moiety” refers to Rc moiety of formula I, and the terms “Rc1 and Rc2 moiety” refer to Rc1 and Rc2 of formula II, when Rc2 is present. According to the present disclosure, Rc1 and Rc2 groups are chelating agents. In other words, the Rc, Rc1 and Rc2 moieties enable chelation of one or more metals by forming a complex.

Each of the Rc, Rc1 and Rc2 moiety may comprise two or more coordination sites. Preferably, the coordination site is a nitrogen or oxygen atom. Advantageously, each of the Rc, Rc1 and Rc2 moiety comprises between 4 and 8 coordination sites, more preferably between 6 and 8 coordination sites and even more preferably each of the Rc, Rc1 and Rc2 moiety comprises 6 coordination sites.

As used herein, the term “coordination site” refers to a single function capable of complexing a metal. For example, an amine function represents a coordination site by the formation of a dative bond between the nitrogen atom and the metal, and a hydroxamic acid function also represents a coordination site by the formation of a dative bond between the oxygen of the carbonyl unit and by a covalent bond with the oxygen of the N-oxide unit the coordination site thus forming a five-membered ring.

In an embodiment, for the functionalized statistic chitosan of formula I, each Rc moiety is independently selected from the group consisting of DOTA (1,4,7,10-tetraazacyclododecane-N,N′,N″,N″-teracetic acid), NOTA (1,4,7-triazacyclononane-1,4,7-triacetic acid), NODAGA (1,4,7-triazacyclononane-1-glutaric-4,7-diacetic acid), DOTAGA (2-(4,7,10-tris(carboxymethyl)-1,4,7,10-tetraazacyclododecan-1-yl) pentanedioic acid), DOTAM (1,4,7,10-tetrakis(carbamoylmethyl)-1,4,7,10-tetraazacyclodecane), NOTAM (1,4,7-tetrakis(carbamoylmethyl)-1,4,7-triazacyclononane), DOTP (1,4,7,10-tetraazacyclododecane 1,4,7,10-tetrakis(methylene phosphonate), NOTP (1,4,7-tetrakis(methylene phosphonate)-1,4,7-triazacyclononane), TETA (1,4,8,11-tetraazacyclotetradecane-N,N′,N″,N″-teracetic acid), TETAM (1,4,8,11-tetraazacyclotetradecane-N,N′,N″,N′″-tetrakis(carbamoyl methyl), DTPA (diethylene triaminopentaacetic acid) Bz-DFO (benzyl deferoxamine) and DFO (deferoxamine), preferably from the group consisting of DOTAGA, Bz-DFO, DFO, DOTAM and DTPA, and more preferably the Rc group is DOTAGA.

In another embodiment, for the functionalized statistic chitosan of formula II, Rc1 and Rc2 are independently selected from the group consisting of DOTA, NOTA, NODAGA, DOTAGA, DOTAM, NOTAM, DOTP, NOTP, TETA, TETAM, DTPA, Bz-DFO and DFO, preferably from the group consisting of DOTAGA, Bz-DFO, DFO, DOTAM and DTPA.

In an embodiment, the chelating agent is selected from the group consisting of:

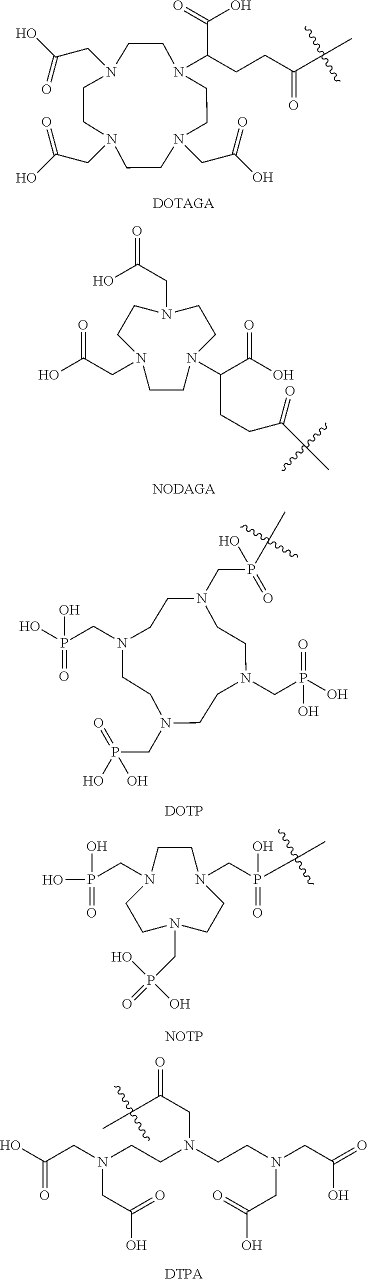

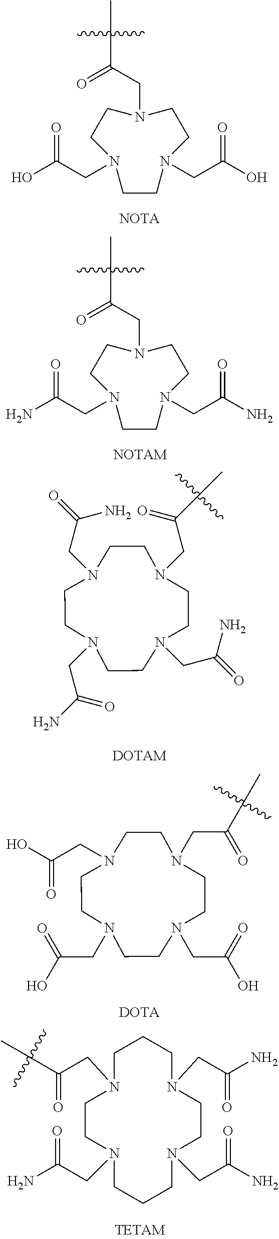

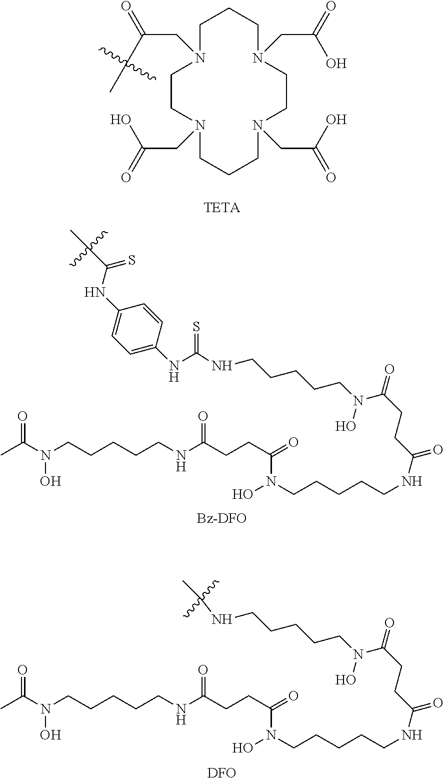 [SMILES: *C(=O)CN1CCCN(CC(=O)O)CCN(CC(=O)O)CCCN(CC(=O)O)CC1 ; *C(=S)Nc1ccc(NC(=S)NCCCCCN(O)C(=O)CCC(=O)NCCCCCN(O)C(=O)CCC(=O)NCCCCCN(O)C(C)=O)cc1 ; *NCCCCCN(O)C(=O)CCC(=O)NCCCCCN(O)C(=O)CCC(=O)NCCCCCN(O)C(C)=O ; "DFO"]

In an embodiment, for the functionalized statistic chitosan of formula II, the group Rc1 is DOTAGA, and preferably, z/y=1.

In another embodiment, for the functionalized statistic chitosan of formula II, the group Rc1 is DOTAGA and the group Rc2 is Bz-DFO.

### Z Linkers (Z, Z1 and Z2)

As used herein, the term “Z linkers” refers to Z linker of formula I, and the terms “Z1 and Z2 linkers” refer to Z1 and Z2 linkers of formula II, when the Z2 binder is present.

The choice of the Z, Z1 and Z2 linkers in formula I and II depends essentially on the Rc, Rc1 and Rc2 moieties and the metal to be chelated. Indeed, for stearic reasons in particular, the Rc, Rc1 and Rc2 moieties may be more or less close to the 6-membered ring of the nitrogen of the glucosamine unit.

In formula I, each Z is independently a linker which is a single bond or a hydrocarbon chain containing between 1 and 12 carbon atoms, said hydrocarbon chain is linear or branched and optionally contains one or more unsaturations and one or more heteroatoms, preferably chosen from nitrogen, oxygen, sulfur and halogens.

In an embodiment, in formula I, each Z is independently selected from the group consisting of: a bond, a linear or branched alkyl chain having between 1 and 12 carbon atoms, and a linear or branched alkenyl chain having between 2 and 12 carbon atoms, said alkyl and alkenyl chains may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR′—C(S)—, —NR′—C(S)—NR, said alkyl and alkenyl chains may be substituted with one or more groups selected from the group consisting of halogen, —OR′, —COOR′, —SR′, —NR′₂, each R′ being independently H or C1-C6 alkyl.

Advantageously, in Formula I, each Z is independently selected from the group consisting of: a bond and a linear or branched alkyl chain having between 1 and 12 carbon atoms, said alkyl chain may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —C(S)NR′—, —NR′—C(S)—NR′, each R′ being independently H or C1-C6 alkyl.

In an embodiment each Z is an alkyl chain having between 1 and 12 carbon atoms.

In another embodiment, each Z is a polyethylene glycol (PEG).

Advantageously, in Formula II, Z1 and Z2 are independently a single bond or a hydrocarbon chain having between 1 and 12 carbon atoms, wherein said chain may be linear or branched and may have one or more unsaturations and may have one or more heteroatoms, preferably selected from nitrogen, oxygen, sulfur, and halogens.

In an embodiment, in formula II, Z1 and Z2 are independently selected from the group consisting of: a bond, a linear or branched alkyl chain having between 1 and 12 carbon atoms, and a linear or branched alkenyl chain having between 2 and 12 carbon atoms, said alkyl and alkenyl chains may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —NR′—C(O)—NR′—, —NR′—C(O)—O—, —O—C(O)NR′, —C(S)NR′—, —NR′—C(S)—, —NR′—C(S)—NR, said alkyl and alkenyl chains may be substituted with one or more groups selected from the group consisting of halogen, —OR′, —COOR′, —SR′, —NR′₂, each R′ being independently H or C1-C6 alkyl.

In an embodiment, in Formula II, Z1 and Z2 are independently selected from the group consisting of: a bond and a straight or branched alkyl chain having between 1 and 12 carbon atoms, wherein said alkyl chain may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —C(S)NR′—, —NR′—C(S)—NR′, each R′ being independently H or C1-C6 alkyl.

In a specific embodiment Z1 and/or Z2 is an alkyl chain having between 1 and 12 carbon atoms.

In another specific embodiment, Z1 and/or Z2 is a polyethylene glycol (PEG).

### Monomeric Units of the Functionalized Statistic Chitosan

The functionalized statistic chitosan in formulae I of the present disclosure is composed of 3 different monomeric units, namely an N-acetylglucosamine type A unit, a glucosamine type B unit and a glucosamine type C unit functionalized by a chelating agent (of the Rc type) linked by a linker (of the Z type) to the nitrogen of the glucosamine.

The functionalized statistic chitosan is statistic polymer. In other words, the sequence of the individual monomer units A, B and type C is random.

The functionalized statistic chitosan in formulae II of the present disclosure is composed of 4 different monomeric units, namely an N-acetylglucosamine type A unit, a glucosamine type B unit and two glucosamine type C unit, namely C1 and C2, functionalized by a chelating agent (of the Rc1 type or Rc2 type) linked by a linker (of the Z1 type or Z2 type) to the nitrogen of the glucosamine.

The functionalized statistic chitosan in formulae II is statistic polymer. In other words, the sequence of the individual monomer units A, B, C1 and C2 is random.

In formulae I and II, x represents the proportion of A units and x is between 0.005 and 0.7, preferably between 0.05 and 0.7, more preferably between 0.2 and 0.6, even more preferably x is between 0.25 and 0.4, typically about 0.3. In an embodiment, x is between 0.025 and 0.075, more preferably between 0.04 and 0.06, typically about 0.05.

In formulae I and II, y represents the proportion of C-type units and y is between 0.01 and 0.7, preferably between 0.05 and 0.2. In an embodiment, y is between 0.03 and 0.2, preferably between 0.05 and 0.1, even more preferably between 0.07 and 0.08, typically about 0.072. In another embodiment, y is between 0.05 and 0.3, more preferably between 0.1 and 0.2, typically about 0.15 or 0.12.

In another embodiment, when the Rc1 and Rc2 of formula II are independently selected from the group consisting of DOTA, NOTA, NODAGA, DOTAGA, DOTAM, NOTAM, DOTP, NOTP, TETA, TETAM, DTPA, Bz-DFO and DFO, preferably from the group consisting of

DOTAGA, Bz-DFO, DFO, DOTAM and DTPA, and y is between 0.05 and 0.3, more preferably between 0.1 and 0.2, typically about 0.12, Rc1 is between 0.06 and 0.08, typically about 0.07 and Rc2 is between 0.04 and 0.06, typically about 0.05.

The rest of the monomer units in formulae I and II are B units. Thus, in formulae I and II, the proportion of B units is equal to 1-x-y.

According to the disclosure, in formulae I and II, the ratio y/x is greater than or equal to 0.05, preferably greater than or equal to 0.15. Indeed, effectiveness of the functionalized statistic chitosan is determined by the number of chelation sites, which is directly related to the number of metals required, for example, for reducing local inflammation induced by and/or inducing a deregulation of metal homeostasis and for reducing oxidative stress, in a subject in need thereof.

To be administered as a solution to a subject in need thereof while being effective, the functionalized statistic chitosan must be soluble at physiological pH, i.e. pH of between 4.8 and 8. For this purpose, the sum of x+y may be greater than or equal to 0.15, preferably greater than or equal to 0.30, more preferably greater than or equal to 0.35.

The combination of the particular ratio between the number of A units and the number of C units and the sum of the proportion of A units and the proportion of C units makes it possible to obtain adequate chelation and solubility allowing the functionalized statistic chitosan to be used in the treatment of diseases characterized by local inflammation induced by and/or inducing a deregulation of metal homeostasis and increased oxidative stress, in a subject in need thereof.

In accordance with the invention, z/y is between 0.5 and 1. In other words, the C-type units may be exclusively units having Z1 as a linker and Rc1 as a chelating agent-bearing group.

The functionalized statistic chitosan has a weight average molecular mass between 100 kDa and 1000 kDa, preferably between 150 kDa and 750 kDa, more preferably between 200 kDa and 600 kDa, even more preferably between 250 kDa and 400 kDa, and even more preferably around 300 kDa.

In an embodiment, the functionalized statistic chitosan is selected from the following functionalized statistic chitosan:
a functionalized statistic chitosan of formula II where z/y=1, Rc1 is DOTAGA and Z1 is a bond;
a functionalized statistic chitosan of formula II where z/y=1, Rc1 is DTPA and Z1 is a bond; and
a functionalized statistic chitosan of formula II where 0.5≤z/y<1, Rc1 is DOTAGA and Z1 is a bond, and Rc2 is Bz-DFO and Z2 is selected from the group consisting of: a bond and a straight or branched alkyl chain having between 1 and 12 carbon atoms, wherein said alkyl chain may be interrupted by one or more C6-C10 aryl groups, and/or by one or more heteroatoms or groups selected from the group consisting of —O—, —S—, —C(O)—, —NR′—, —C(O)NR′—, —NR′—C(O)—, —C(S)NR′—, —NR′—C(S)—NR′, each R′ being independently H or C1-C6 alkyl.

In an embodiment, the functionalized statistic chitosan has the following formula (III):

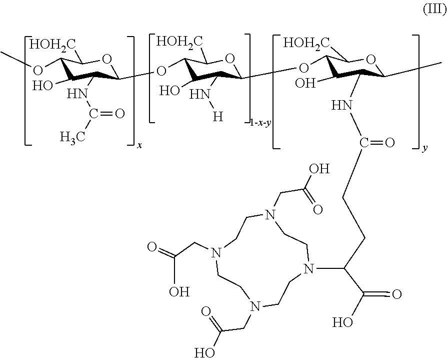

wherein x is between 0.25 and 0.4, typically about 0.3, and y is between 0.05 and 0.2, typically about 0.07.

In another embodiment, the functionalized statistic chitosan has the formula (III), wherein x is between 0.025 and 0.075, more preferably between 0.04 and 0.06, typically about 0.05, and y is between 0.05 and 0.3, more preferably between 0.1 and 0.2, typically about 0.15.

In another embodiment, the functionalized statistic chitosan has the following formula (IV):

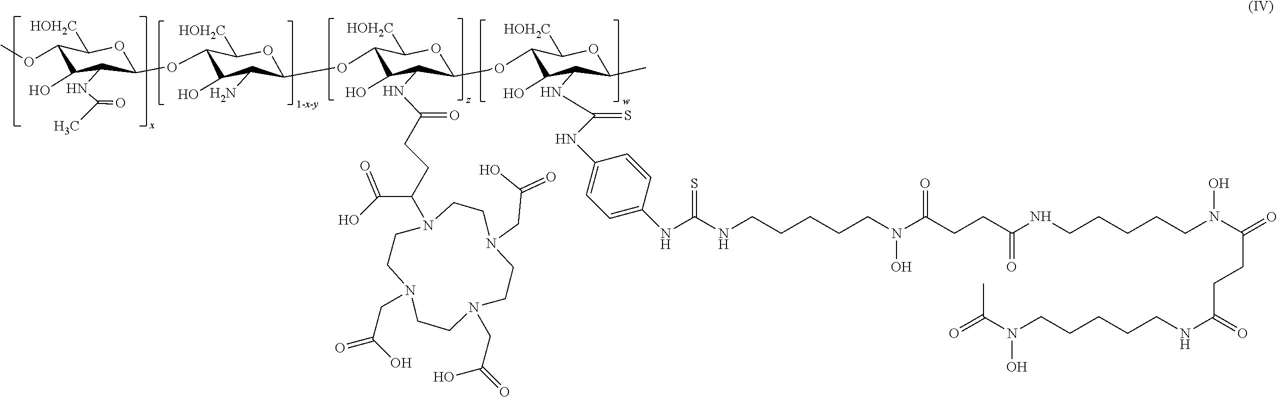

wherein x is between 0.2 and 0.6, more preferably x is between 0.25 and 0.4, typically about 0.3, and y=z+w is between 0.05 and 0.3, more preferably between 0.1 and 0.2, typically about 0.12.

### Synthesis of the Compounds of Formula

The compounds of formulae I and II can be synthesized using the methods disclosed in WO 2022/023677 and in the reference Natuzzi, M., Grange, C., Gréa, T. et al. Feasibility study and direct extraction of endogenous free metallic cations combining hemodialysis and chelating polymer. Sci Rep 11, 19948 (2021).

### Pharmaceutical Composition of the Functionalized Statistic Chitosan

The functionalized statistic chitosan for use may be injected as such or may be formulated in the form with one or more pharmaceutically acceptable excipients.

The pharmaceutical composition may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition in an amount sufficient to provide a therapeutic effect, for example, an improved clinical outcome, such as more frequent complete or partial remissions, or longer disease-free and/or overall survival, or a lessening of symptom severity or other benefit as described in detail herein.

The pharmaceutical composition described herein may be administered to a subject in need thereof by any of several routes that can effectively deliver an effective amount of the compound. In an embodiment the pharmaceutical composition is administered by parenteral route. When the pharmaceutical composition is administered by parenteral route, it may be administered by injection, that is, using a needle (usually a hypodermic needle) and a syringe, or by the insertion of an indwelling catheter. Examples of parenteral administration include intravenous, intramuscular, intranasal, transdermal, submucosal, intrathecal, subcutaneous, intracapsular, intravaginally, intraperitoneally, intrauterine, intraocular, intra-cerebral, intra-bladder, intra-pleural, and intra-articular injection, etc.

A second aspect of the disclosure pertains to the functionalized statistic chitosan of the present disclosure formulated in the form of an injectable solution with one or more pharmaceutically acceptable excipients. Any suitable excipients for injection known to those of ordinary skill in the art in pharmaceutical compositions may be employed in the compositions described herein. For example, such pharmaceutically acceptable excipients may be isotonic sodium chloride, i.e. saline solution, for example NaCl 0.9%; or water, for example ultra-pure water or water for injection; or solution or buffer solution of injectable quality. The person skilled in the art may use NaOH or HCl solution in order to obtain a pH which is appropriate for injection.

In an embodiment, the functionalized statistic chitosan is formulated in the form of an injectable solution with saline solution, preferably NaCl 0.9%. For example, the formulation with saline solution is made so as to obtain an osmolarity inferior or equal to 300 mOsm. In another embodiment, the functionalized statistic chitosan is formulated in the form of an injectable solution with water, preferably ultra-pure water. In another embodiment, the pharmaceutical composition is formulated so as to obtain an injectability with at the most a 27G needle, i.e. capable to be injected with a needle having a gauge of 27G maximum.

In an embodiment, said injectable solution is liquid presenting a viscosity inferior to 5 Pa·s, preferably inferior to 2 Pa·s, more preferably between 0.050 Pa·s and 0.500 Pa·s, even more preferably between 0.075 Pa·s and 0.350 Pa·s, even more preferably around 0.100 Pa·s.

In an embodiment, said injectable solution is liquid and is not in the form of a hydrogel. Indeed, the injectable solution may present a viscosity inferior to 5 Pa·s a pH of between 6.0 and 7.5 and an osmolarity between 50 and 500 mOsm, preferably between 200 and 400 mOsm.

The viscosity is a Newtonian viscosity measured according to the method of rotational rheometry at a regulated temperature (25° C.). Briefly, viscosity is measured with a flow sweep linear study conducted by scanning shear rate from 10⁻¹ to 10³ s⁻¹. The plateau obtained at low shear rates corresponds to the Newtonian viscosity of the sample.

Thanks to these specific properties, the injectable solution once administered in the subject body is stable and will diffuse locally, i.e. not systemically, in said cavity without precipitating.

The functionalized statistic chitosan of the disclosure is formulated in an injectable solution with one or more pharmaceutically acceptable excipients, wherein the concentration of the functionalized statistic chitosan is between 10 g/L and 50 g/L, preferably between 15 g/L and 30 g/L, more preferably between 18 g/L and 25 g/L, even more preferably around 20 g/L. Any suitable excipients known to those of ordinary skill in the art for use in pharmaceutical compositions may be employed in the compositions described herein.

In another embodiment, the injectable solution further comprises one or more additional active ingredients, for example selected from the group consisting of antibodies or classical anti-inflammatory drug. For example, classical anti-inflammatory drug may be either corticosteroids or non-steroidal anti-inflammatory drugs such as NSAID-carboxylic acid (salicylates, flufenanmic acid, indomethacin, diclofenac, ketorolac, dexibuprofen, tiaprofenic acid . . . ) or NSAID-carboxamides/oxicams (piroxicam, meloxicam) or NSAID-sulphonanilides (nimesulide) or NSAID-Diaryl-substituted pyrazoles/furanones (coxibs, celexoxib, etoricoxib, parecoxib, rofecoxib, valdecoxib).

In another embodiment, the injectable solution is formulated to be administered locally, i.e. it is not a systemic injection. In an embodiment, said local injection is an injection in a cavity, preferably said injection is selected from the group consisting of intra-peritoneal, intra-uterine, intra-ocular, intra-cerebral, intra-bladder, intra-pleural, and intra-articular injection, for example intra-synovial injection.

In another embodiment, the injectable solution comprises a combination of functionalized statistic chitosans of the present disclosure, for example the injectable solution comprises a functionalized statistic chitosan of formula III with a functionalized statistic chitosan of formula IV.

### Methods of Treatment Using Said Functionalized Statistic Chitosan

The functionalized statistic chitosan of the present disclosure is for use in the treatment of diseases characterized by local inflammation induced by and/or inducing a deregulation of metal homeostasis and increased oxidative stress, in a subject in need thereof.

As used herein, the term “local” means limited to a certain area in the body of the subject contrary to the systemic term which means not limited to a certain area but extensive in the whole body of the subject. The “certain area” may be a cavity which designates a hollow space, natural or artificial, with a certain volume, closed or not, in the body of the subject.

For example, the cavity may be a cavity already present in the subject body such as the dorsal cavity which comprises the cranial cavity, bounded by the skull and containing the brain, eyes and ears and the spinal cavity, delimited by the vertebral column and containing the spinal cord; the ventral cavity which comprises the thoracic cavity, delimited by the rib cage and containing the lungs and the heart located in the mediastinum; the abdominal cavity, delimited at the top by the rib cage and at the bottom by the pelvis and divided into 2 parts: the peritoneal cavity containing the stomach, intestines, liver, pancreas and gallbladder and the retroperitoneal space containing the kidneys and ureters; and the pelvic cavity, delimited by the pelvis and containing the bladder, the anus and the reproductive system. In a specific embodiment, the cavity may be peritoneal, uterine, ocular, cerebral, bladder, pleural, and articular cavities, for example synovial cavity. Therefore, in this embodiment, the disease characterized by local inflammation of such cavities may be endometriosis, peritonitis, retinal ischemia (including transient and permanent ischemia), glaucoma, age-related macular degeneration, ischemic stroke, intracranial haemorrhage (ICH) including aneurysmal subarachnoid hemorrhage, cystitis including acute and interstitial cystitis, radiation-induced cystitis, osteoarthritis, rheumatoid arthritis, gout, lupus, pleural effusion such as haemothorax.

The cavity may be a cavity not already present in the subject body i.e. a cavity that has been created either naturally or caused by an internal or external event. The internal event may be a blocked blood vessel because of atherosclerosis plug or blood clots, as it can be the case for stroke, or the internal event may be a ruptured blood vessel which causes local internal hemorrhage. This local internal hemorrhage may be traumatic or non-traumatic, i.e. not the result of a trauma, but the result of cardiovascular causes such as high blood pressure, ruptured aneurysm, ruptured tumor or vascular malformation, aortic dissection; digestive causes such as deepening ulcer that has reached a large vessel, Crohn's disease, hemorrhoids, hemorrhagic colic, hiatal hernia; gynecological and obstetrical causes such as endometriosis, IUD contraception, fibroids, heavy menorrhagia, uterine atony (postpartum hemorrhage); primary coagulation disorders such as hemophilia A or B, Willebrand's disease, factor XIII deficiency, hypofibrinogenemia, thrombopathy; secondary coagulation disorders such as hepatic insufficiency, vitamin K deficiency, consumption coagulopathy, circulating anticoagulant; tumoral causes such as colon cancer, bladder cancer (hematuria), uterine cancer, cervical cancer, stomach cancer (hematemesis), lung cancer (hemoptysis), glioblastoma; iatrogenic causes such as taking anticoagulants without monitoring the INR, aspirin, non-steroidal anti-inflammatory drugs. This local internal hemorrhage may be microbleeds. This local internal hemorrhage creates a cavity wherein there are local inflammation and increased oxidative stress. In an embodiment, when the blood vessel ruptures, the local internal hemorrhage will create a dysregulation of iron homeostasis because the iron contained in the red blood cells will be released as a result of the death of the red blood cells. The iron concentration will therefore increase locally, generating inflammation. Iron is also known to activate macrophages and generate reactive oxygen species (ROS) by catalyzing the reactions that produce them, therefore it will also cause increased oxidative stress. Reactive oxygens and nitrogen species, more specifically superoxide and nitrite oxide, have the ability to release copper from caeruloplasmin, the major copper carrier in blood (Fisher et al, 2005). This released copper is redox active and can trigger the production of reactive oxygen species and promote lipid peroxidation (Swain et al, 1994).

Thus, in a specific embodiment, the functionalized statistic chitosan is for use in diseases selected from diseases characterized by local inflammation of one of the following cavities: peritoneal, uterine, ocular, cerebral, bladder, pleural, and articular cavities, for example synovial cavity. Said disease characterized by local inflammation of the peritoneal and/or uterine cavity is selected from the group consisting of endometriosis, peritonitis. Said disease characterized by local inflammation of the ocular cavity is selected from the group consisting of retinal ischemia (including transient and permanent ischemia), glaucoma, age-related macular degeneration. Said disease characterized by local inflammation of the cerebral cavity is selected from the group consisting of ischemic stroke, intracranial haemorrhage (ICH) including aneurysmal subarachnoid hemorrhage. Said disease characterized by local inflammation of the bladder cavity is selected from the group consisting of cystitis including acute and interstitial cystitis, radiation-induced cystitis. Said disease characterized by local inflammation of the articular cavity is selected from the group consisting of osteoarthritis, rheumatoid arthritis, gout, lupus. Said disease characterized by local inflammation of the pleural cavity is selected from the group consisting of pleural effusion such as haemothorax.

In an embodiment, said metal homeostasis is copper, iron, lead, zinc, aluminum, cadmium or manganese homeostasis, preferably copper or iron homeostasis, more preferably iron homeostasis.

In certain aspect, the functionalized statistic chitosan is for use as local injection in said cavity. Preferably said injection is selected from the group consisting of intra-peritoneal, intra-uterine, intra-ocular, for example intra-vitreal, intra-cerebral, for example intra-striatal, intra-bladder, intra-pleural, and intra-articular injection, for example intra-synovial injection. When the cavity is created by local internal hemorrhage, said injection is made at the location of this created cavity.

Thus, the functionalized statistic chitosan for use or the injectable solution of the functionalized statistic chitosan enables chelation of one or more metals by forming a complex thanks to its chelating agent. Consequently, the metal which is free in the body will be captured by the chelating agent so that the functionalized statistic chitosan exerts a direct antioxidant action via its ROS scavenger properties, an indirect antioxidant action by chelating the metal which will no longer be available to catalyze the formation of ROS and to excite macrophages that generate ROS. Besides, thanks to the chelation properties of the functionalized statistic chitosan of the present disclosure, the development of pathogens that could proliferate in the area will also be prevented. Iron is an essential element for both humans and pathogens and bacteria can scavenge iron from their host by various mechanisms including the release of siderophores. Thus, depriving bacteria of iron by using an iron chelator could reduce pathogen proliferation and local infections.

In a preferred embodiment, the metal is iron, and it is the dysregulation of iron homeostasis that induces or is induced by the disease characterized by local inflammation and increased oxidative stress in a subject in need thereof.

In another preferred embodiment, the metal is copper, and it is the deregulation of copper homeostasis that induces or is induced by the disease characterized by local inflammation and increased oxidative stress in a subject in need thereof.

### FIGURES

FIG. 1: Rheological characterization of Newtonian viscosity in a flow diagram displaying the viscosity Eta (Pa·s) vs Shear Rate (1/s) (A) and injectability (force on piston upon injection) with 27G needle (B) of MEX-CD1 formulation at 50 g/L in saline solution before and after sterilization.

FIG. 2: Nociceptive threshold in g (A) and nociceptive scores in % (B) 4 h post-Cyclophosphamide (CYP) injection in Vehicle=Saline, Acetylated Chitosan (DA=30% and Mw=260 kg/mol), MEX-CD1 and MEX-CDDFO1 groups in acute model of cystitis. (##P<0.01 and ###P<0.001)

FIG. 3: Effects of chitosan, MEX-CD1 and MEX-CDDFO1 on CYP-induced bladder inflammation (macroscopic evaluation of bladder wall thickness, oedema and haemorrhage scores) in a rat model of acute cystitis.

FIG. 4: Dosage of IL-1B (A), Monocyte Chemoattractant Protein-1 (MCP-1) (B) and Vascular cell adhesion protein 1 (VCAM-1) (C) expressed in pg/mL (top panel) and as % of variation from saline control group (bottom panel), 4 h after CYP injection in vehicle, acetylated chitosan (DA=30% and Mw=260 kg/mol), MEX-CD1 and MEX-CDDFO1 groups in acute model of cystitis.

FIG. 5: Time-course of MEX-CD1 (30 mg/mL, intra-vesical administration (i.ves)) effects on nociceptive threshold in chronic cystitis rat model, * P<0.05.

FIG. 6: Effects of MEX-CD1 (30 mg/mL, i.ves) on CYP-induced bladder inflammation in a chronic cystitis rat model. Bladder weight expressed in mg (A) and as % of body weight (B), bladder wall thickness (C) and oedema scores (D), after CYP injection (D10) in Vehicle and MEX-CD1 groups. * P<0.05, ** P<0.01 and *** P<0.001

FIG. 7: Nociceptive scores (%) at zone 1 (bladder zone), 2 (colonic zone) and 3 (neutral zone), 4 weeks after surgery in fat (Sham/Vehicle) or uterine horn (ENDO) grafted in rats treated with vehicle or MEX-CD1 (100 mg/kg, 4 treatments), * P<0.05 and ** P<0.01.

FIG. 8: Abdominal electromyographic (EMG) activity in response to vaginal distension in fat (Sham/Vehicle) or uterine horn (ENDO) grafted in rats treated with vehicle or MEX-CD1 (100 mg/kg, 4 treatments). * P<0.05

FIG. 9: T2 images examples obtained by MRI for cortical lesion visualization on D1 in vehicle and MEX-CD1 treated groups (rat model).

FIG. 10: T2* images examples obtained by MRI for haemorrhage visualization on D1 in vehicle and MEX-CD1 treated groups.

FIG. 11: Haemorrhagic lesion volume (A) and lesion volume (B) measured at D1. Boxes show median and quartiles. Whiskers show min and max. Individual values are plotted. Lines indicate the mean. * P<0.05

FIG. 12: Haemorrhagic lesion volume (A) and lesion volume (B) measured at D5. Boxes show median and quartiles. Whiskers show min and max. Individual values are plotted. Lines indicate the mean. * P<0.05

FIG. 13: Neurological score on D1 and D5 after ICH (higher score=more deficits) for control and MEX-CD1 treated group.

FIG. 14: Right induced and left non induced eyes B-wave response at day 7 in a rat model of retinal ischemia.

FIG. 15: Reduction of ferritin staining of rat brains (in % of area stained) at day 5 after ICH with MEX-CD1 treatment (p=0.0029*). Imaging of stained brains was divided in 3 regions of interested (ROI) per hemisphere (D: dorsal, ML: mediolateral and V: ventral).

### EXAMPLES

Hereinafter, the present disclosure is described in more details and specifically with reference to the examples, which however are not intended to limit the present invention.

### Example 1: Synthesis of MEX-CD1 (Chitosane@DOTAGA) and MEX-CDDFO1 (Chitosan@DOTAGA@DFO)

### MEX-CD1

MEX-CD1 synthesis was previously described in WO 2022/023677. Briefly, MEX-CD1 synthesis is composed of three steps.

In the first step, 60 g of chitosan, 4 L of ultra-pure water and 45 mL of glacial acetic acid were introduced in a 10 L-reactor and the mixture was stirred for 16 hours at a pH of 4.5±0.5. A slightly yellowish solution is obtained.

In the second step, 1.2 L of propane-1,2-diol were added to the solution obtained at the end of the previous step and the mixture is stirred for 1 h. Then a solution containing 14 mL of acetic anhydride and 600 mL of propane-1,2-diol is slowly added to the mixture during 10 min to allow a homogenous acetylation of the polymer chain and the reaction media is then stirred for 4 hours. Acetylation degree of 30% (x=0.3) was determined by ¹H RMN by Hirai method (Determination of degree of deacetylation of chitosan by ¹H NMR spectroscopy Asako Hirai, Hisashi Odani & Akio Nakajima, Polymer Bulletin volume 26, pages 87-94 (1991)).

In the third step, 2 L of propane-1,2-diol was added to the previous solution of acetylated chitosan. Then 60 g of DOTAGA anhydride are added and the mixture is stirred for 16 h. After reaction, the solution is diluted by 2 in acetic acid solution at 0.1M and purified using a 100 kDa membrane (Sartocon® Slice 200, PES membrane) with a Sartoflow® Advanced apparatus. Purification is performed at a constant volume (2 VO) towards 15 dia-volumes of 0.1M acetic acid then 10 dia-volumes of water.

### MEX-CDDFO1

MEX-CDDFO1 is double-grafted polymer with DOTAGA and Bz-DFO synthetized from lyophilized MEX-CD1. Briefly, 20 g of lyophilized MEX-CD1 were dissolved in 2 L of ultra-pure water. This solution is stirred at room temperature until complete dissolution. pH is then measured at 6.3. A second solution of p-NCS-Bz-DFO is then prepared by dissolving 3.8 g of p-NCS-Bz-DFO in 380 mL of DMSO. The solution is stirred at room temperature for at least 30 min. Then the solution of MEX-CD1 in water is then poured in a 10 L reactor and 80 mL of ultrapure water is added. Then, 1.2 L of 1,2-propanediol is added under stirring and at controlled temperature (30° C.). 380 mL of DMSO are then added slowly into the reactor. The mixture is stirred at 30° C. for 1 h then the solution of p-NCS-Bz-DFO is slowly added in the reactor using a peristaltic pump at a flow rate of 400 μL/min. The reactor is maintained at 30° C. during the reaction and stirred for 16 h30. The synthetized product is then purified by tangential filtration using a 100 kDa membrane (Sartocon® Slice 200, PES membrane) with a Sartoflow® Advanced apparatus. Briefly, the mixture is diluted by 5 in acetic acid 0.1M then concentrated to reach a dilution factor of 2 compared to the initial volume. Then the product is purified towards acetic acid 0.1M then ultra-pure water and re-concentrated to the initial volume.

### Example 2: Formulation of MEX-CD1 at 20 mg/mL with 8 g/L of NaCl, pH 7

1.007 g of lyophilized MEX-CD1 was solubilized in 45 mL of saline solution (NaCl 0.9%) overnight. pH of the solution was then adjusted to pH 7.0 with 500 μL of 1M NaOH solution. Solution was then completed with ultra-pure water until a total volume of 50.36 mL. Formulation was then stirred for 1 h then sterilized by autoclave (20 min at 121° C.). Osmolarity of the final sterilized formulation was 277 mOsm and pH was measured at 7.1.

### Example 3: Formulation of MEX-CD1 at 10 mg/mL with 7 g/L of NaCl, pH 6

1.00 g of lyophilized MEX-CD1 was solubilized in 95 mL of ultra-pure water overnight. pH of the solution was then adjusted to 6±0.5 (with 1M NaOH solution or 1M HCl solution). Solution was then completed with ultra-pure water until a final volume of 100 mL. Sterilization was performed by autoclave (20 min, 121° C.). pH and osmolarity were measured: pH 6.3, 221 mOsm.

### Example 4: Formulation of MEX-CD1 at 30 mg/mL with 1 g/L of NaCl, pH 6

0.499 g of lyophilized MEX-CD1 was solubilized in 15.5 mL of ultra-pure water. pH was adjusted to 6.0 with 20 μL of 12M HCl solution. Then 537 μL of a saline solution (NaCl) at 30 g/L was added. The formulation was completed with 67.6 μL of ultra-pure water to reach a final formulation volume of 16.1 mL. Osmolarity and pH were measured: 63 mOsm and pH 6.14. The final formulation was sterilized by autoclave (20 min at 121° C.).

### Example 5: Formulation of Viscous Solutions of MEX-CD1

MEX-CD1 can also be formulated at higher concentration, with or without salts. In addition to the previous formulations described in examples 2 to 4, a formulation study was performed. Three polymer concentrations were tested (30, 40, 50 g/L) and three compositions were prepared:
A: MEX-CD1 in ultra-pure water
B: MEX-CD1 with various concentrations of NaCl to reach a final osmolarity around 300 mOsm for all tested concentration
C: MEX-CD1 in saline solution (NaCl 0.9%).

Osmolarity, pH, Newtonian viscosity and the injectability of each formulation were measured before and after sterilisation (by autoclave, 20 min at 121° C.) and are summarized in Table 1.

pH was measured with a calibrated Mettler Toledo SevenCompact PH meter S210 equipped with an InLab Micro Pro-ISM pH electrode with KCl 3 mol/L as reference electrolyte. Osmolarity was measured using a Loser Micro Osmometer MOD200 Plus. Before measurement, the instrument is calibrated with distilled water for zero, 300 mOsm/kg and 900 mOsm/kg standards. Viscosity measurements were conducted on a Thermo Scientific HAAKE RheoStress 600 Sensor Systems using a C35/2° Ti L cone plate geometry. Injectability was defined as the injection force needed for injecting the formulation with a 27G needle, glass prefilable syringue—Hylok SCF 1 ml—with a diameter of 0.63 cm, at a speed of 1 mm·s⁻¹ and was performed as described in Adv. Healthcare Mater. 2020, 9, 1901521 using a Shimadzu AG-X plus force machine equipped with a 10 kN cell. In this example the ease of injection is classified (rated by a panel of participants) regarding the injection force: below 12 N, 5 mL of a substance can easily be injected; between 12 N and 38 N, a considerable effort is needed to inject 5 mL; between 38 N and 64 N a great effort is required and less than 5 mL can be injected; above 64 N, a substance is entirely non-injectable. Thus, according to the results of Table 1, all MEX-CD1 formulations can be injected as injection forces are all below 32 N (Table 1). Specifically, MEX-CD1 formulations at 30 g/L are easily injectable with a 27G needle, glass prefilable syringue—Hylok SCF 1 ml—with a diameter of 0.63 cm, at a speed of 1 mm·s⁻¹. In addition, MEX-CD1 can be easily formulated at pH 6.6 in saline solution (NaCl 0.9%) until 50 g/L.

TABLE 1
Characterization of MEX-CD1 formulations at high concentrations

| | [Polymer] (g/L) | | | | | | | |
|---|---|---|---|---|---|---|---|---|
| | 30 | | | 40 | | | 50 | |
| Formulation number | 30A | 30B | 30C | 40A | 40B | 40C | 50A | 50C |
| [NaCl] (g/L) | 0 | 8.2 | 9 | 0 | 7.8 | 9 | 0 | 9 |

Characterisation before sterilization

| | | | | | | | | |
|---|---|---|---|---|---|---|---|---|
| pH | 6.52 | 6.62 | 6.63 | 6.54 | 6.6 | 6.65 | 6.53 | 6.63 |
| (bulk) | | | | | | | | |
| Osmolarity | 23 | 305 | 319 | 34 | 311 | 350 | 40 | 372 |
| (mean of triplicates, | | | | | | | | |
| in mOsm) | | | | | | | | |
| Newtonian viscosity | 0.313 | 0.327 | 0.294 | 1.224 | 1.170 | 1.148 | 4.105 | 3.925 |
| (Pa · s) | | | | | | | | |
| Injectability (N) | 12.04 | 11.98 | 11.55 | 20.46 | 19.26 | 21.23 | 28.63 | 28.28 |

Characterization after sterilization

| | | | | | | | | |
|---|---|---|---|---|---|---|---|---|
| Newtonian viscosity | 0.233 | 0.275 | 0.268 | 1.192 | 1.163 | 1.300 | 3.564 | 3.759 |
| (Pa · s) | | | | | | | | |
| Injectability (N) | 11.28 | 12.54 | 11.97 | 19.7 | 20.18 | 19.28 | 31.95 | 31.25 |

### Example 6: Other Formulations of MEX-Polymers at 10 mg/mL with 7 g/L of NaCl, pH6

MEX-CDDFO1 (double-grafted polymer with Bz-DFO and DOTAGA) and acetylated chitosan can also be formulated as MEX-CD1 according to example 3. Briefly, 125.2 mg of acetylated chitosan was solubilized in ultra-pure water then 87.7 mg of NaCl was added. After addition of 0.5 mL of ultra-pure water, pH was adjusted to 6.0 with 250 μL of 1M NaOH solution and formulation was completed with ultra-pure water to a final volume of 12.5 mL. Similarly, 203.6 mg of MEX-CDDFO1 polymer was solubilized in 19 mL of ultra-pure water. 143 mg of NaCl was added to the solution, pH was adjusted to 6.0 and formulation was completed with ultra-pure water to a final volume of 20.36 mL. Both formulations were sterilized by autoclave (20 min at 121° C.).

### Example 7: Use of MEX-CD1 Formulation by Intra-Vesical Administration for the Treatment of Acute Cystitis in Rat Model

Acute cystitis was induced by a single intra-peritoneal (i.p.) injection of cyclophosphamide (CYP) at a dose of 150 mg/kg in Sprague-Dawley rats. Control rats (10 rats) received saline solution (0.9% NaCl solution). After CYP injection, 4 groups of 10 rats (see Table 2) were treated with one of the following solutions: saline (vehicle), MEX-CD1 formulation at 10 mg/mL as described in example 3, MEX-CDDFO1 formulation at 10 mg/mL or acetylated chitosan formulation at 10 mg/mL as described in example 6. Treatment consisted in the injection of MexBrain formulation or vehicle at a dose of 500 μL/rat, by intra-vesical administration via a syringe which was connected to the intra-vesical catheter.

TABLE 2
Experimental groups repartition for acute cystitis in a rat-model

| | | | Number |
|---|---|---|---|
| | Injection | Treatment | of |
| Group | (i.p.) | (i.ves.) | rats |
| Model control | Saline | Saline | 10 |
| Substance control | CYP | Saline | 10 |
| Chitosan treated | CYP | Chitosan | 10 |
| MEX-CD1 treated | CYP | MEX-CD1 | 10 |
| MEX-CDDFO1 | CYP | MEX-CDDFO1 | 10 |
| treated | | | |

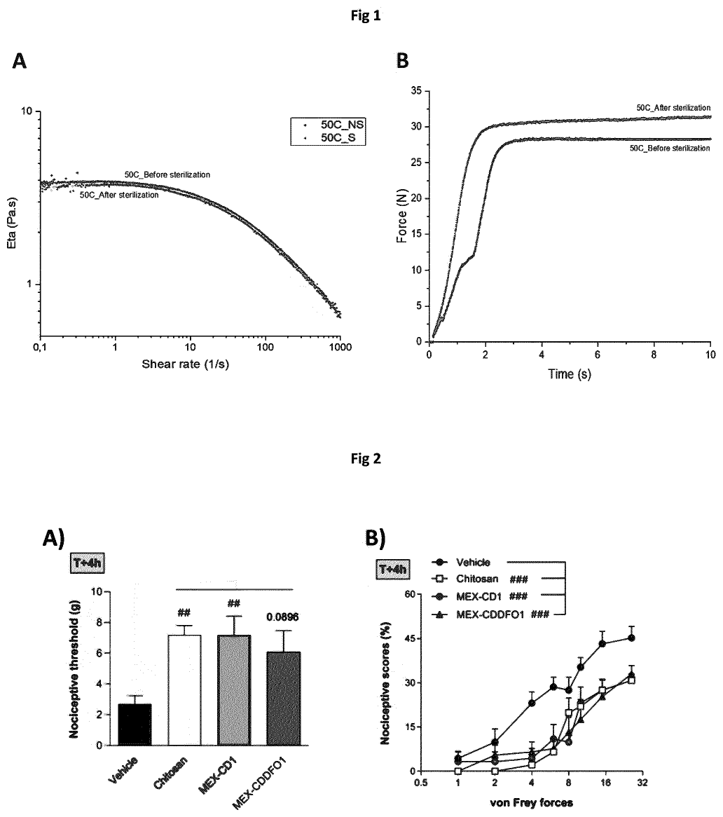

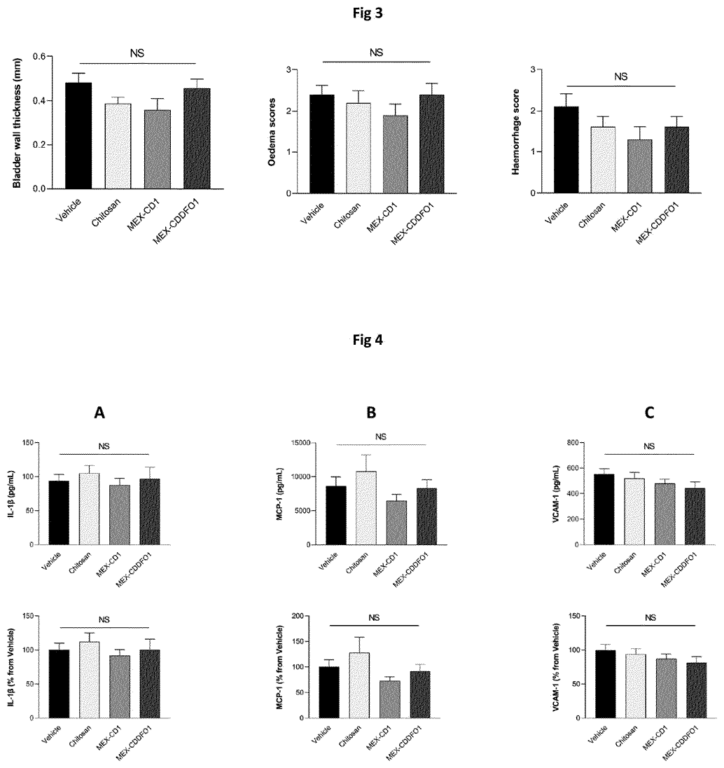

Results showed that injection of chitosan, MEX-CD1 or MEX-CDDFO1 significantly reduced CYP-induced visceral pain evaluated by von Frey testing (FIG. 2). In addition, treatment with MEX-CD1 resulted in a non-significant reduction of macroscopic inflammatory parameters, FIG. 3 shows a slight decrease in bladder wall thickness as well as in oedema scores and haemorrhage scores. A first decrease of several inflammatory markers level in bladder tissue (MCP-1, IL-1B, VCAM-1) was also observed with MEX-CD1 (see FIG. 4). To conclude, MEX-CD1 exhibited anti-nociceptive properties and moderate overall anti-inflammatory response in the acute rat model of CYP-induced cystitis.

### Example 8: Intra-Vesical Injection of MEX-CD1 for the Treatment of Chronic Cystitis in Rat Model

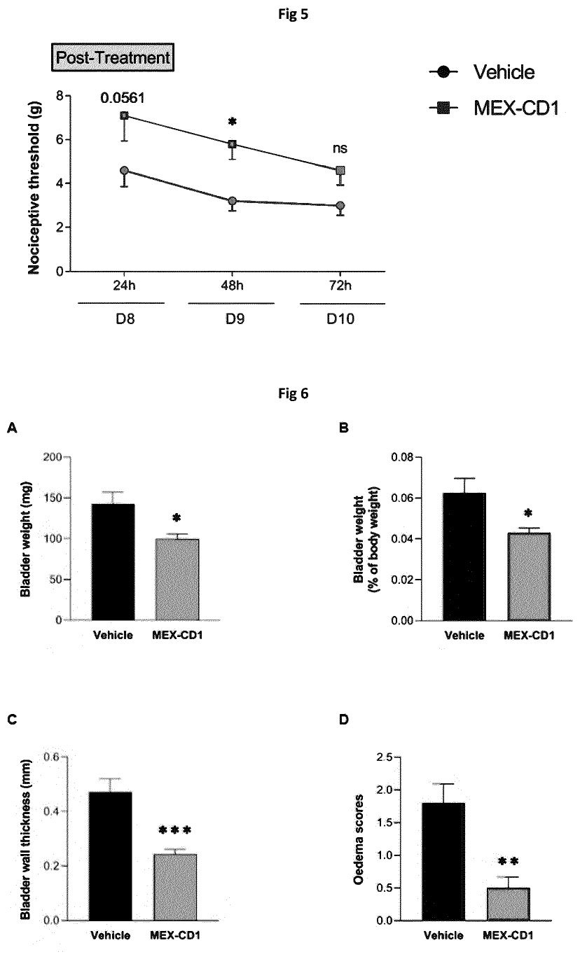

The aim was to evaluate the effects on visceral pain and urinary bladder inflammation of intra-vesical administration (i.ves.) of MEX-CD1 at 30 mg/mL (formulated as described in example 4) in the model of chronic cystitis induced by CYP in Female Sprague-Dawley rats. Chronic cystitis was induced by three intra-peritoneal (i.p.) injections of CYP (DO, D3 and D6) at a dose of 40 mg/kg. The day after the last CYP injection (D7), 500 μL/rat of MEX-CD1 formulation or saline were administered via i.ves. administration as described previously in example 7. Each experimental group (vehicle or MEX-CD1) included 10 rats. Visceral pain was assessed every day after treatment until 72 h (D10), when urinary bladders were collected to assess inflammation. A slight increase of nociceptive threshold was observed after MEX-CD1 instillation, with an effect that was at the limits of the statistical significance level after 24 h, significant after 48 h and above the significance level after 72 h (FIG. 5). Regarding bladder inflammation, 72 h after MEX-CD1 instillation, a significant decrease of bladder weight compared to vehicle was observed. In addition, MEX-CD1 treated group exhibited a significant decrease in bladder wall thickness as well as a reduced oedema score (FIG. 6). Thus, MEX-CD1 presents a marked anti-inflammatory effect in the chronic cystitis model.

### Example 9: Intra-Peritoneal Administration of MEX-CD1 for the Treatment of Endometriosis in a Rat Model

Endometriosis in Female Sprague-Dawley rats was induced by oestradiol treatment and autograft of uterine horn. Sham group underwent the same surgery with fat graft instead of uterine horn. Four weeks after surgery, rats were treated with MEX-CD1 (formulated according to example 2) or vehicle (saline solution) at a dose of 5 mL/kg, which corresponds for MEX-CD1 to a dose of 100 mg/kg. MEX-CD1 group was divided in two cohorts of four rats which received respectively 3 or 4 administrations of MEX-CD1 once a week. Sham group and endometriosis group treated with vehicle were each constituted of 12 rats.

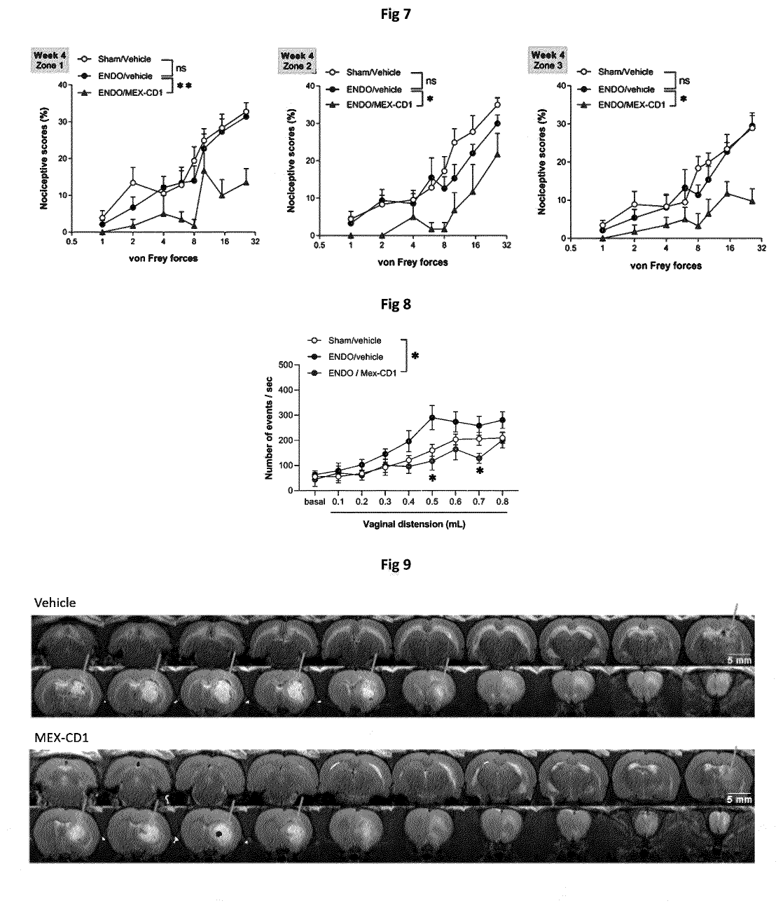

Visceral and vaginal pain was evaluated by von Frey testing and vaginal distension using abdominal electromyography (EMG) recording, respectively. Even if no difference in visceral pain was observed between sham and endometriosis, an interesting decrease of nociceptive score in all tested zones from Von Frey test was observed after four MEX-CD1 treatments compared to vehicle group and sham group (FIG. 7). FIG. 8 shows also a beneficial effect of MEX-CD1 on vaginal pain after 4 treatments as EMG abdominal activity is decreased compared to sham grafted rats and uterine grafted rats treated with vehicle. These results seem to indicate an effect of MEX-CD1 on the reduction of vaginal pain in a rat-model of endometriosis despite the non-significance of the data.

### Example 10: Intra-Cerebral Administration of MEX-CD1 for Improvement of Parenchymal Lesion and Acute Sensorimotor Recovery

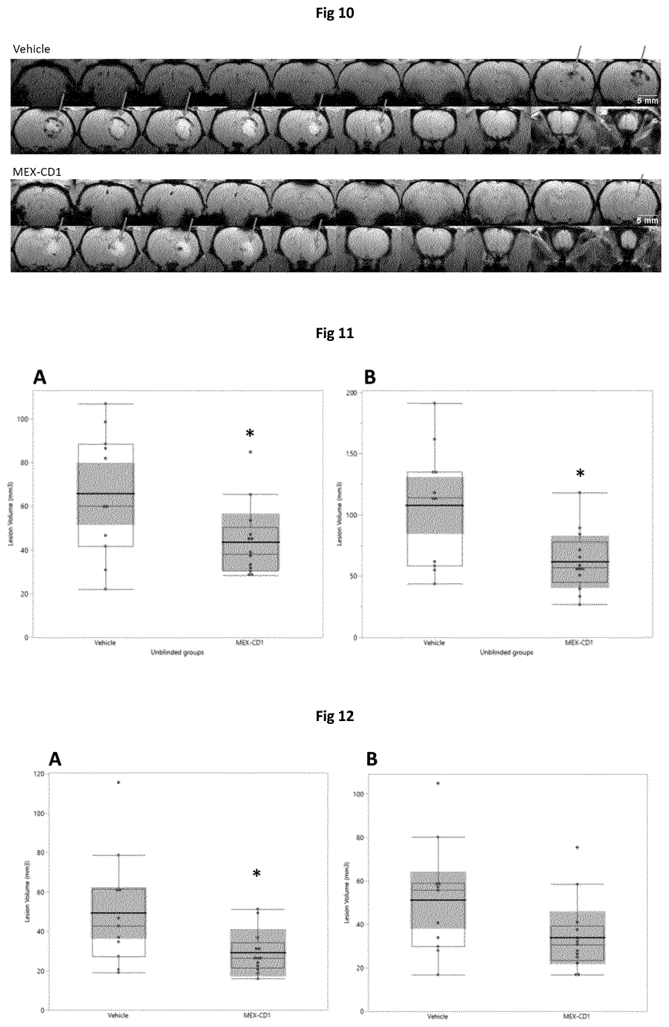

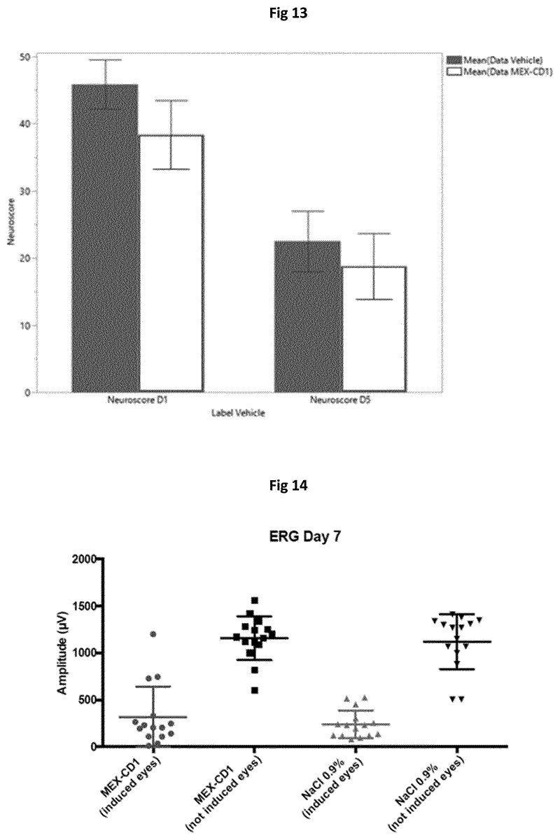

The effect of MEX-CD1 local administration on acute sensorimotor recovery on a rat model of intracerebral haemorrhage was evaluated. Intracerebral haemorrhage (ICH) was induced by bacterial collagenase injection into the striatum in Sprague-Dawley rats. Animals were treated by intra-striatal injection of MEX-CD1 (formulated following example 2) or vehicle (NaCl 0.9%) (1 μL) 30 min after ICH, at a speed of 0.25 μL/min. Haemorrhage and lesion volumes were assessed by MRI at day 1 (D1) and day 5 (D5). T2 imaging allows to estimate cerebral lesion via proton movement alteration and T2* imaging detects deoxyhemoglobin deposits in the cerebral parenchyma, allowing to quantify cerebral haemorrhage volume. In addition, an extended neurological examination was performed at D1 and D5. MRI images at D1 in vehicle and MEX-CD1 treated groups are available in FIG. 9 and FIG. 10. FIG. 11 shows that haemorrhagic lesion volume as well as lesion volume were significantly reduced at D1 in MEX-CD1 treated group compared to vehicle. On D5, haemorrhage volume was still significantly reduced in MEX-CD1 treated animals compared to vehicle and lesion volume was smaller but the difference between groups was not significant (FIG. 12). FIG. 13 shows that all animals presented deficits, assessed by the neuroscore on D1 and D5, with a partial recovery along time. Neuroscores did not evidence significant differential deficits or recovery between groups but MEX-CD1 treated animals tended to have better neuroscore. To conclude, administration of MEX-CD1 had a beneficial effect on haemorrhage and lesion volume from D1 to D5 in a rat model of ICH induced by collagenase injection but did not significantly improve functional recovery. In addition, ferritin staining of rat brains after euthanasia (D5) was significantly reduced in the MEX-CD1 treated group compared to the control group which shows that the polymer has targeted iron, decreasing its storage as ferritin (FIG. 15).

### Example 11: Intravitreal Administration of MEX-CD1 for the Treatment of Transient Retinal Ischemia

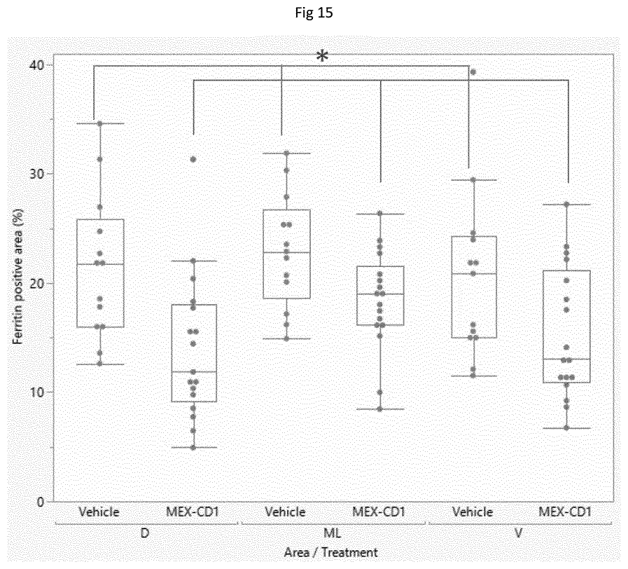

Transient retinal ischemia in Albino Sprague-Dawley rats was induced by increasing intraocular pressure (IOP) to 150 mmHg for 60 minutes and the reperfusion period was initiated by the release of the pressure. 30 rats were randomly divided into two groups of 15 animals. MEX-CD1 (formulated as described in example 2) or control solution (NaCl 0.9%) were administered one day before the induction of retinal ischemia by a single intravitreal injection of 5 μL in the right eye of the animal. Retinal function was evaluated by electroretinography (ERG) at baseline and on day 7 and retinal ganglion cells survival was assessed by immunohistology on day 7 with an anti-BrN3A (Brain-specific homeobox/POU domain protein 3A) labelling. For MEX-CD1 treated group, the B-wave amplitudes in the right induced treated eyes was similar to the one observed in the control group (NaCl) at Day 7 (median value was 205 μV for MEX-CD1 versus 231 μV for control group). However, FIG. 14 shows that three rats from MEX-CD1 treated group have higher B-wave amplitudes in the right induced treated eyes than control group and theses values are close to the ones measured in the left non-induced eyes. In addition, the three same rats (rats 1, 2 and 12 in Table 3) presented higher RGC density in the right induced eye, close to RGC density measured in non-induced eye of control group. Contrarily, three rats in the control group (21, 24 and 30) presented high RGC density after 7 days but only one of them also presented higher B-wave amplitude. Under these experimental conditions, it can be stated that MEX-CD1 displays a first efficacy in rescuing retinal function and protection of the retinal ganglion cells in this rat model of transient retinal ischemia, with three rats out of 15 that seemed to have improved retinal function after this first injection.

TABLE 3
RGC Density on flatmounted retina (Brn3A immunohistology)
at D7 in MEX-CD1 and control group

| | | Retinal Ganglion Cells Density (cells/mm²) | |
|---|---|---|---|
| | Rat | Right Eye | Left Eye |
| Treatment | number | (induced) | (not induced) |
| MEX-CD1 | 1 | 2139 | Not applicable |
| | 2 | 2296 | |
| | 3 | 337 | |
| | 4 | 2046 | |
| | 5 | 1175 | |
| | 6 | 1566 | |
| | 7 | 1140 | |
| | 8 | 770 | |
| | 9 | 680 | |
| | 10 | 948 | |
| | 11 | 1519 | |
| | 12 | 2126 | |
| | 13 | 1549 | |
| | 14 | 1558 | |
| | 15 | 1560 | |
| | Mean | 1427 | |
| | Standard | 583 | |
| | Deviation | | |
| | Median | 1549 | |
| NaCl 0.9% | 16 | 1536 | 2284 |
| | 17 | 1553 | 2420 |
| | 18 | 776 | 2248 |
| | 19 | 1954 | 2249 |
| | 20 | 1037 | 2357 |
| | 21 | 2189 | 2459 |
| | 22 | 1845 | 2403 |
| | 23 | 550 | 2333 |
| | 24 | 2030 | 2167 |
| | 25 | 1237 | 2149 |
| | 26 | 558 | 2160 |
| | 27 | 1933 | 1882 |
| | 28 | 1007 | 2100 |
| | 29 | 1517 | 1966 |
| | 30 | 2268 | 2098 |
| | Mean | 1466 | 2218 |
| | Standard | 578 | 166 |
| | Deviation | | |
| | Median | 1536 | 2248 |

1. A method of reducing inflammation induced by deregulation of metal homeostasis and increased oxidative stress in a subject in need thereof comprising administering to the subject a functionalized statistic chitosan of weight average molecular mass between 100 kDa and 1000 kDa and of formula (I): 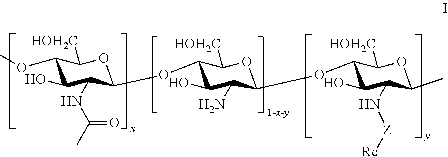
wherein
each Rc is independently a moiety comprising a chelating agent,
each Z is independently a linker which is a single bond or a hydrocarbon chain containing between 1 and 12 carbon atoms, said hydrocarbon chain is linear or branched and optionally contains one or more unsaturations and one or more heteroatoms,
x is between 0.005 and 0.7,
y is between 0.01 and 0.7,
the ratio y/x being greater than or equal to 0.05, and
the sum x+y being greater than or equal to 0.15.

2. The method according to claim 1, wherein each Rc moiety is independently selected from the group consisting of DOTA, NOTA, NODAGA, DOTAGA, DOTAM, NOTAM, DOTP, NOTP, TETA, TETAM, DTPA, Bz-DFO and DFO.

3. The method according to claim 1, wherein the functionalized statistic chitosan is of formula (II): 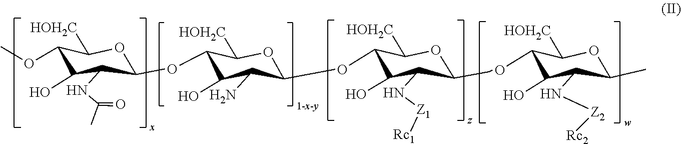
wherein
Rc₁ and Rc₂ are different, and are moiety comprising a chelating agent,
Z₁ and Z₂, identical or different, are linkers which are a single bond or a hydrocarbon chain containing between 1 and 12 carbon atoms, said hydrocarbon is linear or branched and optionally contains one or more unsaturations and one or more heteroatoms,
x is between 0.005 and 0.7,
y=z+w is between 0.01 and 0.7,
the ratio y/x being greater than or equal to 0.05,
the sum x+y being greater than or equal to 0.15, and
z/y is between 0.5 and 1.

4. The method according to claim 3, wherein Rc1 and Rc2 moiety are independently selected from the group consisting of DOTA, NOTA, NODAGA, DOTAGA, DOTAM, NOTAM, DOTP, NOTP, TETA, TETAM, DTPA, Bz-DFO and DFO.

5. The method according to claim 1, having the following formula (III): 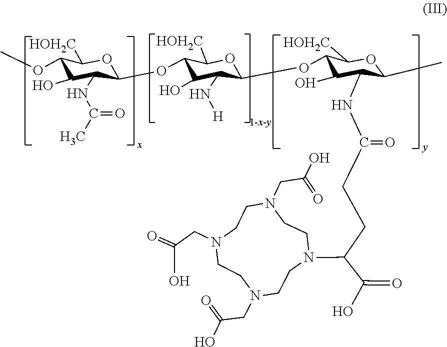
wherein x is between 0.25 and 0.4.

6. The method according to claim 1, wherein said functionalized chitosan is administered by local injection in a cavity, preferably said injection is selected from the group consisting of intra-peritoneal, intra-uterine, intra-ocular, intra-cerebral, intra-bladder, intra-pleural, and intra-articular injection, for example intra-synovial injection.

7. The method according to claim 1, wherein said disease characterized by local inflammation is selected from cystitis, endometriosis, intracranial haemorrhage (ICH), retinal ischemia.

8. The method according to claim 1, wherein said metal homeostasis is iron homeostasis.

9. The method according to claim 1, wherein the injection is not a systemic injection.

10. The method according to claim 1, wherein the functionalized chitosan is administered to the subject at a concentration between 10 g/L and 50 g/L.

11. The method according to claim 1, wherein the functionalized statistic chitosan is in the form of an injectable solution, preferably the injectable solution presents a viscosity inferior to 5 Pa·s, a pH of between 6.0 and 7.5 and an osmolarity of between 50 and 500 mOsm.

12. An injectable solution comprising a functionalized statistic chitosan of weight average molecular mass between 100 kDa and 1000 kDa and of formula (I): 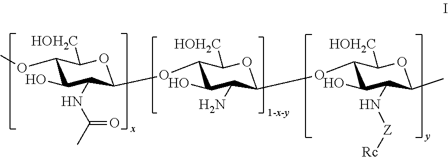
wherein
each Rc is independently a moiety comprising a chelating agent,
each Z is independently a linker which is a single bond or a hydrocarbon chain containing between 1 and 12 carbon atoms, said hydrocarbon chain is linear or branched and optionally contains one or more unsaturations and one or more heteroatoms,
x is between 0.005 and 0.7,
y is between 0.01 and 0.7,
the ratio y/x being greater than or equal to 0.05, and
the sum x+y being greater than or equal to 0.15,
and one or more pharmaceutically acceptable excipients,
wherein the concentration of said functionalized chitosan is of between 10 g/L and 50 g/L.

13. The injectable solution according to claim 12, wherein the concentration of said functionalized chitosan is around 20 g/L.

14. The injectable solution according to claim 10, further comprising one or more additional active ingredients, for example selected from the group consisting of antibodies or classical anti-inflammatory drug, wherein said classical anti-inflammatory drug is either corticosteroids or non-steroidal anti-inflammatory drugs such as NSAID-carboxylic acid or NSAID-carboxamides/oxicams or NSAID-sulphonanilides or NSAID-Diaryl-substituted pyrazoles/furanones.

15. The injectable solution according to claim 10, having the following formula (III): 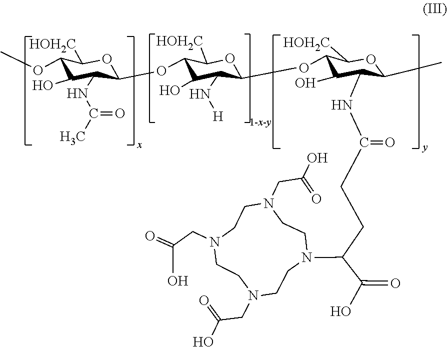
wherein x is between 0.25 and 0.4.
